(19) United States
(12) Patent Application Publication
(10) Pub. No.: US 2023/0307102 A1
(43) Pub. Date: Sep. 28, 2023

(54) Systems and Methods for Automated Identification of ST-Segment Elevation Myocardial Infarction

(71) Applicant: The Board of Trustees of the Leland Stanford Junior University, Stanford, CA (US)

(72) Inventor: Maame Yaa A. B. Yiadom, Palo Alto, CA (US)

(73) Assignee: The Board of Trustees of the Leland Stanford Junior University, Stanford, CA (US)

(21) Appl. No.: 18/191,222

(22) Filed: Mar. 28, 2023

Related U.S. Application Data
(60) Provisional application No. 63/362,033, filed on Mar. 28, 2022.

(51) Int. Cl. G16H 10/60; G16H 50/30; G16H 50/20

(52) U.S. Cl. CPC G16H 10/60; G16H 50/30; G16H 50/20

(57) ABSTRACT

Systems and methods for automated identification of STEMI in accordance with embodiments of the invention are illustrated. One embodiment includes a predictive electronic health record (EHR) system, including a processor, and a memory, the memory containing an EHR management application which configures the processor to obtain preliminary information about a patient via a terminal, store the preliminary information in an EHR, provide the preliminary information to a model trained to predict an emergency medical condition requiring immediate treatment, obtain a likelihood that the patient is suffering from an emergency medical condition from the machine learning model, and provide an alert when the likelihood exceeds a predetermined threshold. In many embodiments, the condition is STEMI, and the preliminary information comprises: age, sex, and at least one chief complaint.

### CROSS-REFERENCE TO RELATED APPLICATIONS

The current application claims the benefit of and priority under 35 U.S.C. § 119(e) to U.S. Provisional Patent Application No. 63/362,033 entitled “Personalized and Predictive Electronic Health Records Systems” filed Mar. 28, 2022. The disclosure of U.S. Provisional Patent Application No. 63/362,033 is hereby incorporated by reference in its entirety for all purposes.

### STATEMENT OF FEDERALLY SPONSORED RESEARCH

This invention was made with government support under HL133477 awarded by the National Institutes of Health. The government has certain rights in the invention.

### FIELD OF THE INVENTION

The present invention generally relates to automated detection of patients at immediate high risk of st-segment elevation myocardial infarction

### BACKGROUND

ST-Segment Elevation Myocardial Infarction (STEMI) is the most severe subtype of acute coronary syndrome (ACS, colloquially referred to as a “heart attack”). STEMI is associated with significant morbidity and mortality. It is most typically caused by a complete thrombotic occlusion developing from an atherosclerotic plaque in an epicardial coronary vessel. Early diagnoses and immediate reperfusion are the most effective ways to limit myocardial ischemia and infarct size.

In clinical settings (hospitals, urgent care, etc.), patient intake often involves clerks without medical education taking the patient's preliminary information. This information is stored in a patient-specific electronic health record (EHR, often called electronic medical records, or “EMRs”) which follow the patient throughout their stay and beyond. EHRs are typically stored in EHR systems which enable clinical staff to access and input medical information about the patient.

### SUMMARY OF THE INVENTION

Systems and methods for automated identification of STEMI in accordance with embodiments of the invention are illustrated. One embodiment includes a predictive electronic health record (EHR) system, including a processor, and a memory, the memory containing an EHR management application which configures the processor to obtain preliminary information about a patient via a terminal, store the preliminary information in an EHR, provide the preliminary information to a model trained to predict an emergency medical condition requiring immediate treatment, obtain a likelihood that the patient is suffering from an emergency medical condition from the machine learning model, and provide an alert when the likelihood exceeds a predetermined threshold.

In another embodiment, the preliminary information comprises: age, sex, and at least one chief complaint.

In a further embodiment, the model is a logistic regression.

In still another embodiment, the logistic regression is log[p/1−p]=intercept+b1*chest_pain+b2*other_ACS_chief_complaints+b3*age_at_visit+b4*gender.

In a still further embodiment, values for constants in the logistic regression are: −5.5<intercept<−5.2; −3.2<b1<−2.9; −0.33<b2<−0.3; 0.03<b3<0.055; and −0.66<b4<−0.62; chest_pain equals 1 when the at least one chief complaint comprises chest pain, chest_pain equals 0 when the at least one chief complaint does not comprise chest pain, and other_ACS_chief_complaints equals 1 when the at least one chief complaint comprises at least one of: chest pain, shortness of breath, weakness, fall, abdominal pain, palpitations, irregular heartbeat, dizziness, altered mental status, nausea, vomiting, syncope, near syncope, abnormal lab or test, arm pain, shoulder pain, hypotension, neck pain, hypertension, heart problem, and cardiac arrest.

In yet another embodiment, the emergency medical condition is ST-elevation myocardial infarction (STEMI).

In a yet further embodiment, the alert is an order for an electrocardiogram.

In another additional embodiment, the predetermined threshold is between 0.0038 and 0.0044.

In yet another again embodiment the predictive model is a machine learning algorithm.

In a further additional embodiment, a method for predicting an emergency medical condition based on preliminary patient information, includes obtaining preliminary information about a patient via a terminal, storing the preliminary information in an EHR, providing the preliminary information to a model trained to predict an emergency medical condition requiring immediate treatment, obtaining a likelihood that the patient is suffering from an emergency medical condition from the machine learning model, and providing an alert when the likelihood exceeds a predetermined threshold.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is an EHR system capable of identifying STEMI in accordance with an embodiment of the invention.

FIG. 2 is an EHR processor in accordance with an embodiment of the invention.

FIG. 3 is a flow chart for a EHR processing to identify STEMI in accordance with an embodiment of the invention.

### DETAILED DESCRIPTION

There are many medical conditions which require immediate diagnosis and treatment for a reasonable chance of survival. For example, over 56 million patients visit emergency departments (EDs) each year for acute coronary syndrome (ACS) evaluation. All need an electrocardiogram (ECG) within a critical 10-minute window of arrival to diagnose the most time-sensitive and life-threatening form of ACS: ST-elevation myocardial infarction (STEMI). Every 1-minute reduction in time to ECG yields approximately a 1.24-minute reduction in time to STEMI treatment. However, 37% percent of STEMI patients do not receive an ECG within the 10-minute window, and are twice as likely to die within 1 week than those receiving an ECG within the 10-minute window.

Systems and methods described herein leverage electronic health records to automatically identify and flag to medical staff that a patient is at risk for a life-threatening, time-critical condition. While the below description is made with reference to STEMI identification among those with ACS, as can be readily appreciated, similar approaches can be taken with respect to any life-threatening, time-sensitive medical condition. Indeed, in a variety of embodiments, different modules can be implemented in an EHR system that have specific predictive capabilities for specific medical conditions. A typical workflow for a given patient intake involves a clerk (often without a medical education) taking the patient's preliminary information. Preliminary information typically includes age, sex, race, ethnicity, and the chief complaint (i.e. the reason for the visit).

This preliminary information is typically stored in a patient-specific electronic health record (EHR) which is managed by an electronic health record system. Electronic health record systems described herein can immediately process the preliminary information about a patient upon ingest and predict a likelihood that the patient is currently or about to suffer from a life-threatening, time-critical condition like STEMI. If the likelihood exceeds a threshold, then the clerk and/or medical professionals can be immediately alerted so testing can begin immediately. In numerous embodiments, a machine learning model is trained and implemented to perform the likelihood calculations. In some embodiments, the model is a logistic regression. However, any number of different predictive models can be used as appropriate to the requirements of specific applications of embodiments of the invention. In various embodiments, other information is ingested and used as part of the likelihood calculation including (but not limited to): how the patient arrived at the hospital (e.g. helicopter, ambulance, walk-in); who ingested the data (clerk, nurse, etc.); whether or not the patient is known to the hospital system; associated EHR data already in the system; etc.

In various embodiments, systems and methods described herein can correctly identify 93% or more of heart attack patients compared to approximately 79% correct identifications made by human hospital staff. While the below provides discussion with respect to STEMI identification, in many embodiments, different models for different diseases classes can be implemented and run during ingestion. EHR systems capable of identifying STEMI are discussed below.

### EHR Systems

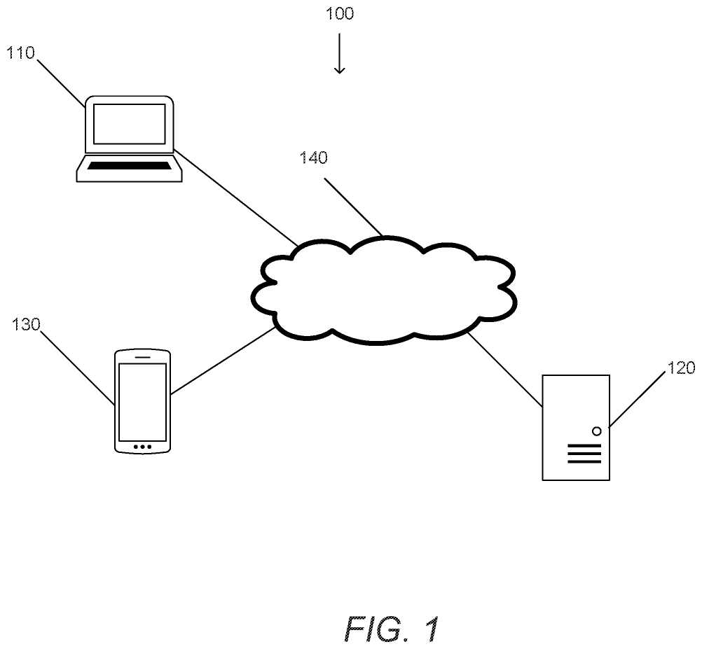

EHR systems are repositories for patient information which is collected almost immediately on patient arrival. When a patient enters, a clerk collects at least the name, age, sex, and chief complaint and enter it into an EHR for the patient via a terminal. This data can be near-instantaneously processed to produce a prediction of whether or not the patient is suffering from STEMI. Turning now to FIG. 1, an EHR system capable of identifying STEMI in accordance with an embodiment of the invention is illustrated.

System 100 includes a terminal 110. Terminals are any computer interface such as (but not limited to) desktop computers, laptops, tablet computers, smart phones, and/or any other device capable of enabling input for creation of EHRs as appropriate to the requirements of specific applications of embodiments of the invention. Terminals can be used by clerks or others doing patient intake to create EHRs. System 100 further includes an EHR processor 120. In numerous embodiments, the EHR processor is implemented at least in part using the terminal. In various embodiments, the EHR processor is implemented using a server, a server cluster, and/or a cloud computing platform. The system 100 further includes alerting devices 130. Alerting devices are any mobile device which can flag to medical professionals that an incoming patient requiring an emergency ECG. Alerting devices can be smart phones, pagers, distributed display screens, and/or any other device capable of issuing alerts as appropriate to the requirements of specific applications of embodiments of the invention.

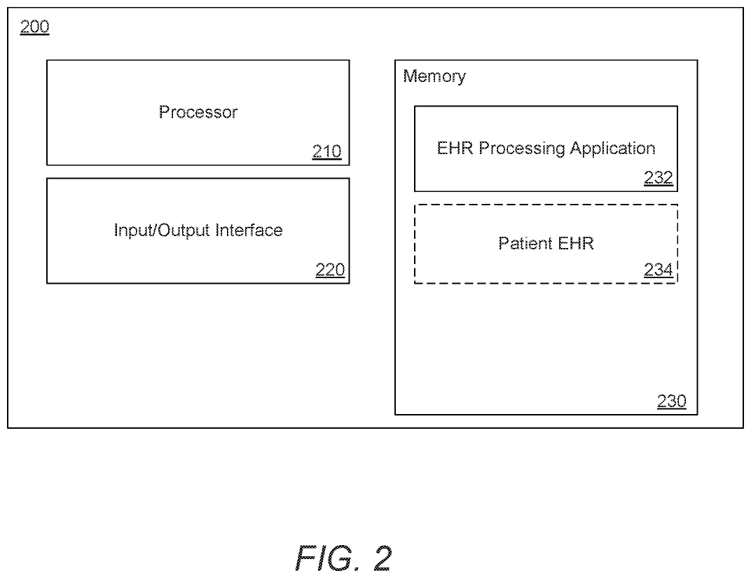

While a particular system architecture is illustrated in FIG. 1, as can readily be appreciated, different computing frameworks can be used depending on the needs of the medical site. Turning now to FIG. 2, an EHR processor in accordance with an embodiment of the invention is illustrated. EHR processor 200 includes a processor 210. Processors are any logic circuits capable of carrying out computation processes including (but not limited to), central processing units (CPUs), graphics processing units (GPUs), application-specific integrated circuits (ASICs), field-programmable gate arrays (FPGAs), and/or any other processing circuitry as appropriate to the requirements of specific applications of embodiments of the invention. EHR processor further includes an input/output (I/O) interface 220. The I/O interface is capable of transmitting and receiving information to other computing devices and peripheral devices using wired and/or wireless connections.

EHR processor 200 further includes a memory 230. Memory is a storage medium capable of storing machine-readable instructions and data. Memory can be implemented using volatile and/or non-volatile memory. Memory 230 stores an EHR processing application 232. EHR processing applications can configure the processor to carry out various computational processes described herein. In various embodiments, the memory 230 variously stores a patient EHR 234 for a particular patient to be processed. EHR processors can be implemented in numerous different ways without departing from the scope or spirit of the invention. In various embodiments, the EHR processor can be distributed across a cloud computing platform. EHR processing to identify STEMI is discussed in further detail below.

### Identifying STEMI

In many medical organizations, patient intake at the minimum involves getting the age, sex, and chief complaint (sometimes called “arrival complaint”) for the patient. It is then up to the clerk or an additional reviewer to decide in what order patients will be seen by doctors. However, if a patient is suffering from STEMI, intervention needs to be applied immediately to have an acceptable prognosis. Humans, especially those that are not medical professionals, have demonstrated an inability to correctly identify and trigger emergency protocols for STEMI patients with a high degree of accuracy. Models described herein can intake age, sex, and chief complaint, and give a sufficiently accurate prediction of whether or not a patient is suffering from STEMI.

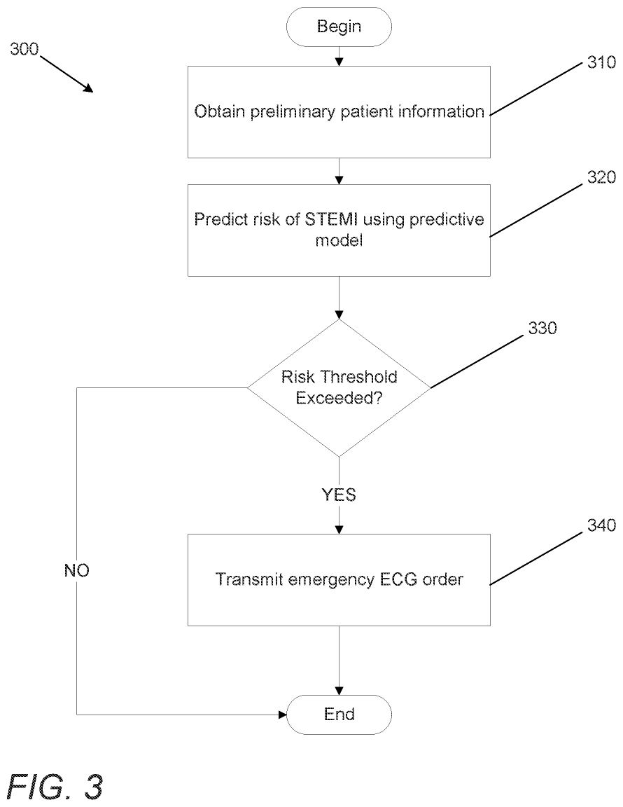

Turning now to FIG. 3, a process for identify STEMI patients based on preliminary health information from an EHR in accordance with an embodiment of the invention is illustrated. Process 300 includes obtaining (310) preliminary patient information which at least includes age, sex, and chief complaint. In numerous embodiments, other information may be obtained as well such as (but not limited to) race, ethnicity, prior conditions, medications, and/or any other information including those found in preexisting EHRs for the patient as appropriate to the requirements of specific applications of embodiments of the invention. In many embodiments, the chief complaint is selected from a predetermined set of complaints. Chief complaints can include any number of different complains. Example chief complaints that can implicate STEMI include (but are not limited to): chest pain, shortness of breath, weakness, fall, abdominal pain, palpitations, irregular heartbeat, dizziness, altered mental status, nausea, vomiting, syncope, near syncope, abnormal lab or test, arm pain, shoulder pain, hypotension, neck pain, hypertension, heart problem, and cardiac arrest.

A risk of STEMI is then predicted (320) using a model. In numerous embodiments, the model is a logistic regression model which provides a likelihood value. In many embodiments, the model is:

Log[p/1−p]=intercept+b1*chest_pain+b2*other_ACS_chief_complaints+b3*age_at_visit+b4*gender

where chest_pain and other_ACS_chief_complaints equal 1 for yes, and 0 for no, and gender equals 1 for female and 0 for male. In numerous embodiments, values for the constants in the following ranges: −5.5<intercept<−5.2; −3.2<b1<−2.9; −0.33<b2<−0.3; 0.03<b3<0.055; and −0.66<b4<−0.62. In various embodiments, value for the constants are as follows: intercept=−5.3668; b1=−3.0571; b2=−0.3169; b3=0.045; and b4=−0.6438. The resulting probability can be exponentiated to calculate the odds of STEMI, and the risk probability can be computed as odds/(1+odds).

If the risk threshold is exceeded (330), an emergency order for an ECG for the patient is transmitted (340) and the patient is immediately moved to treatment. In numerous embodiments, the risk threshold is between 0.0038 and 0.0044. In some embodiments, the risk threshold is 0.0041. However, the risk threshold can be moved depending on the risk tolerance and overall medical situation as a whole for a given institution as appropriate to the requirements of specific applications of embodiments of the invention. Emergency orders may immediately be paged to alerting devices. In some embodiments, patient information is immediately transferred to data recorders connected to ECG devices so that data can be immediately entered.

Although specific methods of automated identification of ST-segment elevation myocardial infarction are discussed above, many different methods can be implemented in accordance with many different embodiments of the invention. For example, in various embodiments, a machine learning model can be trained to identify STEMI patients based on preliminary health information instead of, or in conjunction with, a logistic regression model. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A predictive electronic health record (EHR) system, comprising:
a processor; and
a memory, the memory containing an EHR management application which configures the processor to:
obtain preliminary information about a patient via a terminal;
store the preliminary information in an EHR;
provide the preliminary information to a model trained to predict an emergency medical condition requiring immediate treatment;
obtain a likelihood that the patient is suffering from an emergency medical condition from the predictive model; and
provide an alert when the likelihood exceeds a predetermined threshold.

2. The predictive EHR system of claim 1, wherein the preliminary information comprises: age, sex, and at least one chief complaint.

3. The predictive EHR system of claim 2, wherein the model is a logistic regression.

4. The predictive EHR system of claim 3, wherein the logistic regression is log[p/1−p]=intercept+b1*chest_pain+b2*other_ACS_chief_complaints+b3*age_at_visit+b4*gender.

5. The predictive EHR system of claim 4, wherein values for constants in the logistic regression are: −5.5<intercept<−5.2; −3.2<b1<−2.9; −0.33<b2<−0.3; 0.03<b3<0.055; and −0.66<b4<−0.62;
chest_pain equals 1 when the at least one chief complaint comprises chest pain;
chest_pain equals 0 when the at least one chief complaint does not comprise chest pain; and
other_ACS_chief_complaints equals 1 when the at least one chief complaint comprises at least one of: chest pain, shortness of breath, weakness, fall, abdominal pain, palpitations, irregular heartbeat, dizziness, altered mental status, nausea, vomiting, syncope, near syncope, abnormal lab or test, arm pain, shoulder pain, hypotension, neck pain, hypertension, heart problem, and cardiac arrest.

6. The predictive EHR system of claim 1, wherein the emergency medical condition is ST-elevation myocardial infarction (STEMI).

7. The predictive EHR system of claim 6, wherein the alert is an order for an electrocardiogram.

8. The predictive EHR system of claim 1, wherein the predetermined threshold is between 0.0038 and 0.0044.

9. The predictive EHR system of claim 2, wherein the model is a machine learning model.

10. A method for predicting an emergency medical condition based on preliminary patient information, comprising:
obtaining preliminary information about a patient via a terminal;
storing the preliminary information in an EHR;
providing the preliminary information to a model trained to predict an emergency medical condition requiring immediate treatment;
obtaining a likelihood that the patient is suffering from an emergency medical condition from the machine learning model; and
providing an alert when the likelihood exceeds a predetermined threshold.

11. The method of claim 10, wherein the preliminary information comprises: age, sex, and at least one chief complaint.

12. The method of claim 11, wherein the model is a logistic regression.

13. The method of claim 12, wherein the logistic regression is log[p/1−p]=intercept+b1*chest_pain+b2*other_ACS_chief_complaints+b3*age_at_visit+b4*gender.

14. The method of claim 13, wherein values for constants in the logistic regression are:
−5.5<intercept<−5.2; −3.2<b1<−2.9; −0.33<b2<−0.3; 0.03<b3<0.055; and −0.66<b4<−0.62;
chest_pain equals 1 when the at least one chief complaint comprises chest pain;
chest_pain equals 0 when the at least one chief complaint does not comprise chest pain;
other_ACS_chief_complaints equals 1 when the at least one chief complaint comprises at least one of: chest pain, shortness of breath, weakness, fall, abdominal pain, palpitations, irregular heartbeat, dizziness, altered mental status, nausea, vomiting, syncope, near syncope, abnormal lab or test, arm pain, shoulder pain, hypotension, neck pain, hypertension, heart problem, and cardiac arrest.

15. The method of claim 10, wherein the emergency medical condition is ST-elevation myocardial infarction (STEMI).

16. The method of claim 15, wherein the alert is an order for an electrocardiogram.

17. The method of claim 10, wherein the predetermined threshold is between 0.0038 and 0.0044.

18. The method of claim 10, wherein the model is a machine learning model.
